(19) United States
(12) Patent Application Publication
(10) Pub. No.: US 2011/0136216 A1
(43) Pub. Date: Jun. 9, 2011

(54) DIAGNOSTIC KIT FOR RESPIRATORY DISEASE

(75) Inventors: Kyung-Hyun KIM, Daejeon (KR); Chul Huh, Daejeon (KR); Chil-Seong Ah, Daejeon (KR); Gun-Yong Sung, Daejeon (KR)

(73) Assignee: Electronics and Telecommunications Research Institute, Daejeon (KR)

(21) Appl. No.: 12/902,442

(22) Filed: Oct. 12, 2010

(30) Foreign Application Priority Data
Dec. 9, 2009 (KR) 10-2009-0121682

(51) Int. Cl. C12M 1/34

(52) U.S. Cl. 435/287.2

(57) ABSTRACT

The present invention relates to a diagnostic kit for a respiratory disease that enables a preprocessing process with respect to a sample to be performed within a diagnostic kit when diagnosing respiratory disease using the diagnostic kit, and thereby enables all the operations starting from collecting the sample to verifying a diagnosis result to be automatically performed within the diagnostic kit. The diagnostic kit for a respiratory disease may perform a field diagnosis at anywhere without being restricted to a specific location, and even a general user may also easily obtain an accurate diagnosis result.

### CROSS REFERENCE TO RELATED APPLICATIONS

This application claims priority to Korean Patent Application No. 10-2009-0121682 filed on Dec. 9, 2009, the entire contents of which are herein incorporated by reference.

### BACKGROUND OF THE INVENTION

1. Field of the Invention

The present invention relates to a diagnostic kit for a respiratory disease.

2. Description of the Related Art

Currently, a most widely used medical diagnostic kit may be a pregnancy diagnostic kit and a blood glucose measurement kit for measuring blood glucose. These disposable diagnostic kits basically employ two technologies to verify a pregnancy or to measure blood glucose. A first technology indicates a technology of migrating blood or urine to a sensing portion in a liquid form using a capillary phenomenon. A second technology indicates a technology that may react blood, urine, and the like reaching a diagnosis or measurement available portion and thereby visually identify the result with unaided-eyes using various colorimetry, for example, a fluorescent substance, a dye, a nano-particle, and the like, or indicate the result as a change in minute current or voltage occurring in an electrode using an electromechanical scheme.

In the case of a diagnostic kit for diagnosing epidemic respiratory disease such as novel swine-origin influenza A (H1N1) and the like, the diagnostic kit may be used after a process of collecting and preprocessing body fluid such as sputum, nasal discharge, and the like, using a cotton swab, prior to using the diagnostic kit.

### SUMMARY OF THE INVENTION

When a separate preprocessing process with respect to a sample is required before using a medical diagnostic kit, a field diagnosis that is a greatest advantage of the diagnostic kit may become obsolete. Also, in this case, it may be difficult for a general user to use the diagnostic kit or to obtain an accurate diagnosis result.

Therefore, there is an object of the present invention to provides a diagnostic kit for a respiratory disease that enables a preprocessing process with respect to a sample to be performed within a diagnostic kit when diagnosing a respiratory disease using the diagnostic kit, and thereby enables all the operations starting from collecting the sample to verifying a diagnosis result to be automatically performed within the diagnostic kit.

According to an exemplary embodiment of the present invention, there is provided a diagnostic kit for a respiratory disease, including a substrate, an expired air collecting unit, a micro fluidic channel, and a reactor, wherein: the expired air collecting unit includes a porous micro bead disposed on the substrate to collect, as liquid, a material included in an expired air; the micro fluidic channel is connected to the expired air collecting unit to transfer the collected liquid to the reactor; and the reactor includes an antibody-marker conjugate that is combinable with a lipid bilayer decomposition of a virus fixed in a dried state to an inner wall or a floor of the reactor to be connectable with the liquid transferred via the micro fluidic channel, and protein of the virus exposed by the decomposition.

In a diagnostic kit for a respiratory disease according to embodiments of the present invention, without a separate preprocessing process with respect to a sample, all the operations starting from collecting the sample to verifying a diagnosis result may be automatically performed within the diagnostic kit. Accordingly, the diagnostic kit for a respiratory disease according to embodiments of the present invention may perform a field diagnosis at anywhere without being restricted to a specific location, and even a general user may also obtain an accurate diagnosis result and thus may conveniently use the diagnostic kit.

### BRIEF DESCRIPTION OF THE DRAWINGS

FIG. 1 is a plan view illustrating a structure of a diagnostic kit for a respiratory disease according to an exemplary embodiment of the present invention; and

FIG. 2 is a cross-sectional view illustrating a structure of a diagnostic kit for a respiratory disease according to an exemplary embodiment of the present invention.

### DETAILED DESCRIPTION OF THE PREFERRED EMBODIMENTS

Hereinafter, the exemplary embodiments of the present invention will be described in detail with reference to the accompanying drawings. Herein, the detailed description of a related known function or configuration that may make the purpose of the present invention unnecessarily ambiguous in describing the present invention will be omitted.

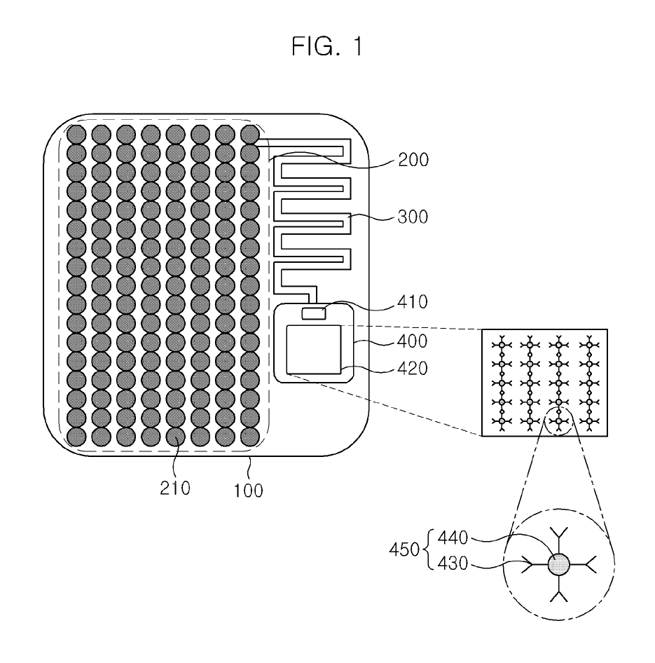

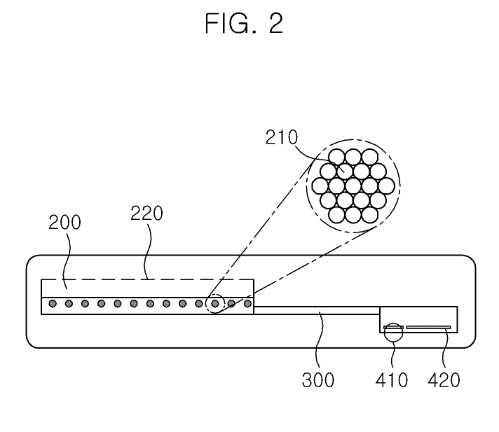

As shown in FIGS. 1 and 2, the present invention relates to a diagnostic kit for a respiratory disease, including a substrate 100, an expired air collecting unit 200, a micro fluidic channel 300, and a reactor 400.

The expired air collecting unit 200 may be disposed on the substrate 100 and include a porous micro bead 210 to collect, as liquid, a material included in an expired air.

The micro fluidic channel 300 may be connected to the expired air collecting unit 200 to transfer the collected liquid to the reactor 400.

The reactor 400 may include an antibody-marker conjugate 450 that may be combinable with a lipid bilayer decomposition 410 of a virus fixed in a dried state to an inner wall or a floor of the reactor 400 to be connectable with the liquid transferred via the micro fluidic channel 300, and protein(antigen) of the virus exposed by the decomposition.

In the present invention, a material of the substrate 100 is not particularly restricted and may employ a material generally used in this art. For example, a plastic material may be used for the substrate 100.

In the present invention, the porous micro bead 210 included in the expired air collecting unit 200 functions to collect, as liquid, a material included in expired air exhaled by a subject. The porous micro bead 210 may include pores having various sizes from a few micro meters to millions of micro meters, and a bead surface may have a hydrophilic property. The bead surface may be coated using a hydrophilic polymer so that the porous micro bead surface may have the hydrophilic property. A representative example of the hydrophilic polymer may include protein such as casein and BSA.

The expired air collecting unit 200 may have a floor structure where the liquid collected from the expired air may be gathered into a portion where the expired air collecting unit 200 is connected to the micro fluidic channel 300.

The porous micro bead 210 may include a plastic polymer such as polyethylene terephthalate (PET), polyether sulfone (PES), cyclo olefin copolymer (COC), polymethyl methacrylate (PMMA), and poly(methyl 2-methylpropenoate), and an oxide material such as Al₂O₃, and SiO₂, and the like, or may include a combination of a polymer family and an oxide family.

In the present invention, the micro fluidic channel 300 functions to transfer, to the reactor 400, the liquid collected by the expired air collecting unit 200 due to a capillary phenomenon, and may be formed of the same material as a material of the substrate 100 and may be formed in an engraved shape within the substrate 100.

A plurality of micro fluidic channels 300 may be formed to effectively transfer, to the reactor 400, the liquid collected by the expired air collecting unit 200 within a short period of time, and an inner surface of the micro fluidic channel 300 may have a hydrophilic property. To enable the inner surface of the micro fluidic channel 300 to have the hydrophilic property, the inner surface may be coated with a hydrophilic polymer. A representative example of the hydrophilic polymer may include protein such as casein and BSA.

In the present invention, a lipid bilayer decomposition 410 of the virus included in the reactor 400 may be fixed in a dried state to the inner wall or the floor of the reactor 400 so as to be well connectable with the liquid collected from the expired air, transferred via the micro fluidic channel 300. A lipid bilayer of the virus within the liquid may be automatically decomposed according to the above contact and thus a separate preprocessing process may not be required.

Materials generally used in this art may be used as the lipid bilayer decomposition of the virus without restrictions. A representative example may include a material of a detergent family. Triton X-100, Tween 20, and the like are on the market as the material of the detergent family.

A scheme for diagnosing a respiratory disease using a known diagnostic kit may include separately performing, outside the diagnostic kit, a process of decomposing a lipid bilayer of a virus by collecting a body fluid such as sputum, nasal discharge, and the like, using a cotton swab, and diagnosing the respiratory disease by applying a resulting material (protein of the virus of which the lipid bilayer is decomposed) to the diagnostic kit. Accordingly, an equipment capable of performing the process of decomposing the lipid bilayer of the virus may be separately required. In a place where it is difficult to prepare the equipment, a field diagnosis may be nearly impossible. Also, even though the equipment is available, it may be difficult to expect a general user to obtain accurate data through the above process.

A diagnostic kit of the present invention enables the process of decomposing the lipid bilayer of the virus, which is separately performed in the art, to be performed within the diagnostic kit, and thereby enables a field diagnosis of a respiratory disease. Even a general user may also easily and accurately diagnose a respiratory disease.

As described above, when decomposing the lipid bilayer of the virus, protein (antigen) of the virus may be externally exposed and thereby be captured by an antibody 430 included in the antibody-marker conjugate 450 pre-fixed to the reactor 400. The protein of the virus may be nucleocapsid protein (NP). The antibody 430 may be a nucleocapsid protein (NP) antibody.

In the above, a marker 440 may use a nano-particle generating a color or having a coloring function to be visually identifiable with unaided-eyes, a fluorescent substance, a metal nano-particle generating an electrical signal, and the like.

A representative nano-particle generating the color may be a gold nano-particle.

The diagnostic kit of the present invention may further include, in the reactor 400, a secondary antibody fixed to easily identify the antibody-marker conjugate 450 combined with the protein (antigen) of the virus. The secondary antibody may be fixed to the floor of the reactor 400 to have a predetermined shape such as one straight line, two straight lines, and the like.

In the above, the secondary antibody functions to be combined with protein (antigen) included in the combination of the protein of the virus and the antibody-marker conjugate 450 and to thereby fix the combination.

Also, the diagnostic kit of the present invention may further include, in the reactor 400, a pair of electrodes for generating an electrical signal.

The diagnostic kit of the present invention may enhance a sensitivity to detect even a low concentration of virus included in a patient's expired air by changing a size of the marker 440 and a number of markers 440, or a material of the marker 440, or by adjusting a reactivity of the antibody 430.

The diagnostic kit of the present invention may be mountable to a disposable mask in addition to a general diagnostic kit form to thereby diagnose the respiratory disease and as a result it may be very convenient. Also, when a radio frequency identification (RFID) chip is installed to the diagnostic kit of the present invention, it is possible to monitor nationwide inspection results. Therefore, the inspection results are predicted to be used as important data for prevention and disease control. A flexible polymer device or a small ultra-slim semiconductor device may be used as the RFID chip.

Hereinafter, a method for diagnosing a respiratory disease using a diagnostic kit for a respiratory disease according to an exemplary embodiment of the present invention will be described using an example.

Initially, expired air may be blown into an expired air hole 220 of the expired air collecting unit 200 of the diagnostic kit for respiratory disease of the present invention during a predetermined period of time (ex., 10 to 20 minutes). A component included in the expired air collected into the expired air collecting unit 200 according to the above process may be collected as liquid based on a micro pore of the porous micro bead 210. The collected liquid may be gathered in one place of the expired air collecting unit 200. The collected liquid may be transferred to the reactor 400 through a plurality of micro fluidic channels 300. The liquid transferred to the reactor 400 may contact with the lipid bilayer decomposition 410 of the virus fixed in the dried state on the inner wall or the floor of the reactor 400. In this instance, when the virus is included in the liquid, the liquid bilayer of the virus may be decomposed. The liquid contacting with the lipid bilayer decomposition 410 is migrated to a portion 420 where the antibody-marker conjugate 450 is fixed within the reactor 400. In this instance, when protein (antigen) of the virus is included in the collected liquid, the protein (antigen) may be combined with the antibody 430 included in the conjugate 450. In this instance, when the marker 440 is a nano-particle or a fluorescent substance generation a color, a plurality of combinations of the protein (antigen) and the conjugate 450 may be cohered to show a color visually identifiable with unaided-eyes.

In this case, when the secondary antibody capable of fixing the combination is fixed in a predetermined shape (ex., one straight line or two straight lines) within the reactor 400, the combination may be combined with the secondary antibody to show a color in a predetermined shape.

Also, when the marker 440 is a metal nano-particle, it is possible to determine the existence of virus by detecting an electrical signal generated by an electrode provided in the reactor 400.

While the present invention has been shown and described in connection with the exemplary embodiments, it will be apparent to those skilled in the art that modifications and variations can be made without departing from the spirit and scope of the invention as defined by the appended claims.

What is claimed is:

1. A diagnostic kit for a respiratory disease, comprising a substrate, an expired air collecting unit, a micro fluidic channel, and a reactor, wherein:
the expired air collecting unit is disposed on the substrate and comprises a porous micro bead to collect, as liquid, a material included in an expired air;
the micro fluidic channel is connected to the expired air collecting unit to transfer the collected liquid to the reactor; and
the reactor comprises an antibody-marker conjugate that is combinable with a lipid bilayer decomposition of a virus fixed in a dried state to an inner wall or a floor of the reactor to be connectable with the liquid transferred via the micro fluidic channel, and protein of the virus exposed by the decomposition.

2. The diagnostic kit for a respiratory disease according to claim 1, wherein the porous micro bead comprises a pore of a micro size, and a bead surface has a hydrophilic property.

3. The diagnostic kit for a respiratory disease according to claim 2, wherein a surface of the porous micro bead is processed using protein to have the hydrophilic property.

4. The diagnostic kit for a respiratory disease according to claim 2, wherein the porous micro bead comprises at least one selected from a group consisting of polyethylene terephthalate (PET), polyether sulfone (PES), cyclo olefin copolymer (COC), polymethyl methacrylate (PMMA), poly(methyl 2-methylpropenoate), Al₂O₃, and SiO₂.

5. The diagnostic kit for a respiratory disease according to claim 1, wherein an inner portion of the micro fluidic channel has a hydrophilic property.

6. The diagnostic kit for a respiratory disease according to claim 1, wherein the lipid bilayer decomposition of the virus corresponds to a material of a detergent family.

7. The diagnostic kit for a respiratory disease according to claim 1, wherein the protein of the virus corresponds to nucleocapsid protein (NP).

8. The diagnostic kit for a respiratory disease according to claim 1, wherein in the antibody-marker conjugate, a marker comprises at least one selected from a group consisting of a nano-particle generating a color to be visually identifiable with unaided-eyes, a fluorescent substance, and a metal nano-particle generating an electrical signal.

9. The diagnostic kit for a respiratory disease according to claim 8, wherein the nano-particle generating the color corresponds to a gold nano-particle.

10. The diagnostic kit for a respiratory disease according to claim 1, wherein the reactor further comprises a pre-fixed secondary antibody to fix a combination of the protein of the virus and the antibody-marker conjugate.
